(19) United States
(12) Patent Application Publication
(10) Pub. No.: US 2023/0241295 A1
(43) Pub. Date: Aug. 3, 2023

(54) IODINATED COMPOUNDS AND HYDROGELS FORMED FROM SAME

(71) Applicant: Boston Scientific Scimed, Inc., Maple Grove, MN (US)

(72) Inventors: Joseph Thomas Delaney, JR., Minneapolis, MN (US); Yen-Hao Hsu, Shrewsbury, MA (US); Tatyana Dyndikova, Minneapolis, MN (US)

(73) Assignee: Boston Scientific Scimed, Inc., Maple Grove, MN (US)

(21) Appl. No.: 18/103,932

(22) Filed: Jan. 31, 2023

Related U.S. Application Data
(60) Provisional application No. 63/305,792, filed on Feb. 2, 2022.

(51) Int. Cl. A61L 31/14; A61L 31/04; A61M 5/19; C07C 233/66

(52) U.S. Cl. CPC A61L 31/145; A61L 31/041; A61M 5/19; C07C 233/66

(57) ABSTRACT

In some aspects, the present disclosure pertains to systems for forming hydrogels that comprise (a) a first composition that comprises a polyiodinated polyamino compound that comprises a polyamino moiety linked to a polyiodinated aromatic moiety by an amide group or ester group and (b) a second composition that comprises a reactive multi-arm polymer that comprises a plurality of hydrophilic polymer arms having reactive end groups that are reactive with amino groups of the polyiodinated polyamino compound. Other aspects of the present disclosure pertain to medical hydrogels and methods of making medical hydrogels that are based on such compositions. Further aspects of the present disclosure pertain to methods of making polyiodinated polyamino compounds.

### CROSS REFERENCE TO RELATED APPLICATIONS

This application claims benefit of U.S. Provisional Patent Application Ser. No. 63/305,792, filed on Febr. 2, 2022, the disclosure of which is incorporated herein by reference.

### FIELD

The present disclosure relates to iodinated compounds, to hydrogels formed from iodinated compounds, and to methods of making and using iodinated compounds and hydrogels, among other aspects. The iodinated compounds of the present disclosure are useful, for example, in forming hydrogels for various biomedical applications.

### BACKGROUND

Bioerodible injectable hydrogels are an emerging class of materials having a variety of medical uses. As one specific example, in the case of SpaceOAR®, a long-term bioerodible injectable hydrogel based on star polyethylene glycol (PEG) polymers end-capped with reactive ester end groups reacting with lysine oligomers to form crosslinked hydrogels, such products are used to create or maintain space between tissues in order to reduce side effects of off-target radiation therapy. See “Augmenix Announces Positive Three-year SpaceOAR Clinical Trial Results,” Imaging Technology News, Oct. 27, 2016.

More recently, hydrogels in which some of the star PEG branches are functionalized with 2,3,5-triiiodobenzamide (TIB) groups have imparted enhanced radiopacity. As a specific example, Augmenix has developed TraceIT® Hydrogel, a bioerodible injectable hydrogel synthetic hydrogel consisting primarily of water and iodinated cross-linked star PEG that is visible under CT, cone beam, ultrasound and MR imaging and is useful as a tissue marker (e.g., for targeted radiation therapy). See “Augmenix Receives FDA Clearance to Market its TraceIT® Tissue Marker,” Business Wire Jan. 28, 2013. TraceIT® hydrogel remains stable and visible in tissue for three months, long enough for radiotherapy, after which it is absorbed and cleared from the body. Id.

Although TraceIT® hydrogel is iodinated as it contains 2,3,5 triiodobenzoate groups, it is not visible on planar x-ray imaging, because the concentration of the 2,3,5 triiodobenzoate groups in the hydrogel is limited by the hydrophobicity of such groups. More generally, in hydrogels in which some of the star PEG branches are functionalized with 2,3,5-triiiodobenzamide groups, an upper limit exists to how many of these groups can be added before it impacts the ability to form a smooth, consistent hydrogel. This solubility limit is in effect a limit on the amount of radiocontrast achievable with this strategy. The 2,3,5-triiiodobenzamide groups need to be added to the PEG prior to reactive functionalization, adding complexity to the star-PEG manufacturing process and resulting in increased product cost, persistence, and difficulties in product quality control. Furthermore, each 2,3,5-triiiodobenzamide group added occupies one arm of the star polymer, reducing its capacity for crosslinking. To overcome this, lower molecular weight star PEG's can be used, but this is at the cost of a lower melting point, which can make storage and shipping a challenge. Finally, star PEG labeled with 2,3,5-triiiodobenzamide end groups often show discoloration from thermal degradation. While this doesn't impact their functionality, this is a cosmetic defect that would be preferably avoided.

There is a continuing need in the biomedical arts for additional hydrogels, for precursors of such hydrogels, for methods of making such hydrogels and precursors, for methods of using such hydrogels and precursors, and for systems for forming such hydrogels, among other needs.

### SUMMARY

In some aspects, the present disclosure pertains to systems for forming a hydrogel. The systems comprise (a) a first composition that comprises a polyiodinated polyamino compound that comprises a polyamino moiety linked to a polyiodinated aromatic moiety by an amide group or ester group and (b) a second composition that comprises a reactive multi-arm polymer that comprises a plurality of hydrophilic polymer arms having reactive end groups that are reactive with amino groups of the polyiodinated polyamino compound.

In some embodiments, which can be used in conjunction with the above aspects, the polyiodinated aromatic moiety comprises a residue of an amino-substituted polyiodinated aromatic compound. In some of these embodiments, the residue of the amino-substituted polyiodinated aromatic compound comprises a monocyclic or multicyclic aromatic moiety that is substituted with (a) an amino group, (b) a plurality of iodine groups and (c) one or a plurality of hydrophilic functional groups.

In some embodiments, which can be used in conjunction with the above aspects, the polyiodinated aromatic moiety comprises a monocyclic or multicyclic aromatic moiety that is substituted with (a) a plurality of iodine groups and (b) one or a plurality of hydrophilic functional groups.

In some embodiments, which can be used in conjunction with any of the above embodiments, the monocyclic or multicyclic aromatic moiety is selected from benzene and naphthalene and wherein the hydrophilic functional groups comprise hydroxyalkyl groups.

In some embodiments, which can be used in conjunction with the above aspects and embodiments, the polyiodinated aromatic moiety is a 1,3-substituted-2,4,6-triiodobenzene moiety in which a substituent at each of the 1- and 3-positions comprises a dihydroxyalkyl group.

In some embodiments, which can be used in conjunction with the above aspects and embodiments, the polyamino moiety comprises a residue of a carboxyl-substituted polyamino compound that comprises a carboxyl group and the polyamino moiety. In some of these embodiments, the carboxyl-substituted polyamino compound is selected from a polylysine compound, a carboxyl-substituted carboxyl-terminated poly(allyl amine) compound, a carboxyl-terminated polyvinylamine compound, and a carboxyl-terminated chitosan compound.

In some embodiments, which can be used in conjunction with the above aspects and embodiments, the polyamino moiety comprises a plurality of —(CH₂)ₓ—NH₂ groups where x is 0, 1, 2 3, 4, 5 or 6. In some of these embodiments, the plurality of —(CH₂)ₓ—NH₂ groups are disposed along a polymeric moiety for example, selected from a polyamide moiety, a polyalkylene moiety, and a polysaccharide moiety, among others.

In some embodiments, which can be used in conjunction with the above aspects and embodiments, the hydrophilic polymer arms comprise one or more hydrophilic monomers selected from ethylene oxide, N-vinyl pyrrolidone, oxazolines, hydroxyethyl acrylate, hydroxyethyl methacrylate, PEG methyl ether acrylate, PEG methyl ether methacrylate, or PNIPAAM.

In some embodiments, which can be used in conjunction with the above aspects and embodiments, the reactive end groups are linked to the hydrophilic polymer arms by a hydrolysable ester.

In some embodiments, which can be used in conjunction with the above aspects and embodiments, the reactive end groups are electrophilic groups. For example, the electrophilic groups may be selected from imidazole esters, imidazole carboxylates, benzotriazole esters, or imide esters, among others.

In some embodiments, which can be used in conjunction with the above aspects and embodiments, hydrophilic polymer arms extend from a polyol residue.

In some embodiments, which can be used in conjunction with the above aspects and embodiments, the system further comprises a delivery device. In some of these embodiments, the delivery device may comprise a first reservoir that contains the first composition and a second reservoir that contains the second composition, wherein during operation of the delivery device, the first and second compositions may be dispensed from the first and second reservoirs, whereupon the first and second compositions interact and crosslink with one another to form the hydrogel. In some embodiments, the first and second reservoirs comprise syringe barrels.

In some aspects, the present disclosure pertains to medical hydrogels that are formed by reaction of the first composition and the second composition of a system in accordance with any of the above aspects and embodiments.

In some aspects, the present disclosure pertains to methods of making medical hydrogels that comprise reacting the first composition with the second composition of a system in accordance with any of the above aspects and embodiments. In some of these aspects, the reaction occurs spontaneously at room temperature or body temperature.

In some aspects, the present disclosure pertains to methods of making polyiodinated polyamino compounds, which comprise (a) forming an amide linkage between an amino group of an amino-substituted polyiodinated aromatic compound and a carboxyl group of a protected carboxyl-substituted polyamino compound in which the amino groups are protected and (b) the deprotecting the protected amino groups. For example, the amino-substituted polyiodinated aromatic compound may be a protected amino-substituted polyiodinated aromatic compound that comprises a monocyclic or multicyclic aromatic moiety that is substituted with (a) the amino group, (b) a plurality of iodine groups and (c) one or a plurality of hydrophilic functional groups that comprise an acetal-protected dihydroxyalkyl group.

In other aspects, the present disclosure pertains to methods of making polyiodinated polyamino compounds, which comprise (a) forming an ester linkage between hydroxyl group of a hydroxyl-substituted polyiodinated aromatic compound and a carboxyl group of a protected carboxyl-substituted polyamino compound in which the amino groups are protected and (b) the deprotecting the protected amino groups.

### BRIEF DESCRIPTION OF THE DRAWINGS

FIGS. 1, 2, 3A and 3B schematically illustrate a method of making a polyiodinated polyamino compound in accordance with an embodiment of the present disclosure.

FIG. 4 schematically illustrates a method of making a hydrogel composition in accordance with an embodiment of the present disclosure.

### DETAILED DESCRIPTION

In some aspects of the present disclosure, a radiopaque crosslinked hydrogel is provided that comprises a crosslinked reaction product of (a) a polyiodinated polyamino compound and (b) a reactive multi-arm polymer that comprises a plurality of reactive end groups that are reactive with the amino groups of the polyiodinated polyamino compound. Unless indicated otherwise, as used herein the prefix “poly” means two or more.

In some aspects of the present disclosure, a system is provided that comprises (a) a first composition that comprises a polyiodinated polyamino compound and (b) a second composition that comprises a reactive multi-arm polymer that comprises a plurality of reactive end groups that are reactive with the amino groups of the polyiodinated polyamino compound.

Such a system is advantageous, for example, in that iodine functionality, and thus radiopacity, is provided by the polyiodinated polyamino compound that acts as a crosslinker for the multi-arm polymer. This allows reactive end groups to be provided on each of the polymer arms, thereby maximizing the crosslinking capacity of the multi-arm polymer, without sacrificing radiopacity.

In some aspects, the present disclosure pertains to polyiodinated polyamino compounds (compounds that comprise a plurality of iodine groups and a plurality of amino groups) which are useful, for example, as crosslinking agents.

In various embodiments, the polyiodinated polyamino compounds of the present disclosure comprise a polyamino moiety and a polyiodinated aromatic moiety.

In various embodiments, the polyiodinated polyamino compounds of the present disclosure comprise a residue of a carboxyl-substituted polyamino compound and a residue of an amino-substituted polyiodinated aromatic compound. Such polyiodinated polyamino compounds may be formed by an amidation reaction in which the carboxyl group of the carboxyl-substituted polyamino compound is reacted with the amino group of the amino-substituted polyiodinated aromatic compound to form an amide bond between the two residues.

In various embodiments, the polyiodinated polyamino compounds of the present disclosure comprise a residue of a carboxyl-substituted polyamino compound and a residue of a hydroxyl-substituted polyiodinated aromatic compound. Such polyiodinated polyamino compounds may be formed by an esterification reaction in which the carboxyl group of the carboxyl-substituted polyamino compound is reacted with a hydroxyl group of the hydroxyl-substituted polyiodinated aromatic compound to form an ester bond between the two residues.

In various embodiments, the polyiodinated polyamino compounds of the present disclosure comprise a polyamino moiety having a plurality (two, three, four, five, six, seven, eight, nine, ten or more) amino groups.

For example, the polyamino moiety may comprises a plurality (two, three, four, five, six, seven, eight, nine, ten or more) of —(CH₂)ₓ—NH₂ groups where x is 0, 1, 2 3, 4, 5 or 6. In some of these embodiments, the polyamino moiety may comprises a plurality of —(CH₂)ₓ—NH2 groups disposed along a polymeric moiety (defined herein as a moiety comprising 2, 3, 4, 5, 6, 7, 8, 9, 10 or more monomer residues). In some embodiments, the polymeric moiety may be selected from a polyamide moiety, a polyalkylene moiety, or a polysaccharide moiety, among others.

As previously indicated, in some embodiments, the polyamino moiety of the polyiodinated polyamino compounds may correspond to a residue of a carboxyl-substituted polyamino compound (a compound comprising a carboxyl group and a plurality of amino groups). Examples of carboxyl-substituted polyamino compounds include polylysines (e.g., dilysine, trilysine, tetralysine, pentalysine, etc.) and carboxyl-terminated polyamines such as carboxyl-terminated poly(allyl amine), carboxyl-terminated poly(vinyl amine), or carboxyl-terminated chitosan.

In various embodiments, the polyiodinated polyamino compounds of the present disclosure comprise a polyiodinated aromatic moiety having a plurality (two, three, four, five, six, seven, eight, nine, ten or more) iodine groups.

For example, the polyiodinated aromatic moiety may comprise a monocyclic or multicyclic aromatic structure that is substituted with (a) a plurality of iodine groups (e.g., two, three, four, five, six, seven, eight, nine, ten or more iodine groups) and (b) one or a plurality of hydrophilic functional groups (e.g., one, two, three, four, five, six or more hydrophilic functional groups).

The monocyclic or multicyclic aromatic structure may be selected, for example, from monocyclic aromatic structures such as those based on benzene and multicyclic aromatic structures such as those based on naphthalene, among others.

The one or the plurality of hydrophilic functional groups may comprise, for example, hydroxyalkyl groups such as C₁-C₄-hydroxyalkyl groups (e.g., C₁-C₄-monohydroxyalkyl groups, C₁-C₄-dihydroxyalkyl groups, C₁-C₄-trihydroxyalkyl groups, C₁-C₄-tetrahydroxyalkyl groups, etc.), among others. The hydroxyalkyl groups may be linked to the monocyclic or multicyclic aromatic structures directly or through any suitable linking moiety, which may be selected, for example, from amide groups, amine groups, ether groups, ester groups, or carbonate groups, among others.

In certain embodiments, the polyiodinated aromatic moiety may comprise a 1,3-substituted-2,4,6-triiodobenzene group, wherein a substituent at each of the 1- and 3-positions comprises a hydrophilic functional group, for example, a hydroxyalkyl group, which may be selected from those described above and which may be linked to the benzene structure directly or through any suitable linking moiety. In a particular example, the 1,3-substituted-2,4,6-triiodobenzene group may be an N,N′-bis(hydroxyalkyl)-2,4,6-triiodobenzene-1,3-dicarboxamide group, for instance, an N,N′-bis(C₁-C₄-hydroxyalky)-2,4,6-triiodobenzene-1,3-dicarboxamide group. The 1,3-substituted-2,4,6-triiodobenzene group, may in turn, be linked through the 5-position to a remainder of the polyiodinated polyamino compound through any suitable linking moiety, including an amide linkage, an amine linkage, an ester linkage, a carbonate linkage, or an ether linkage. In certain embodiments, the iodinated aromatic moiety may comprise a 1,3-(C₁-C₄-hydroxyalkyl-substituted)-2,4,6-triiodobenzene group, where the hydroxyalkyl groups are linked to the benzene structure through an amide linkage, and the iodinated aromatic moiety may be linked through the 5-position to a remainder of the polyiodinated polyamino compound through an amide group.

As previously indicated, in some embodiments, the polyiodinated aromatic moiety of the polyiodinated polyamino compounds may correspond to a residue of an amino-substituted polyiodinated aromatic compound.

For example, the amino-substituted polyiodinated aromatic compound may comprise a monocyclic or multicyclic aromatic structure that is substituted with a plurality of iodine groups, one or a plurality of hydrophilic functional groups such as those described above, and an amino group. For example, in some embodiments, the polyiodinated polyamino compound may comprise a residue of a 5-amino-1,3-substituted-2,4,6-triiodobenzene compound, wherein a substituent at each of the 1- and 3-positions comprises a hydrophilic functional group, for example, a hydroxyalkyl group, which may be selected from those described above and which may be linked to the benzene structure directly or through any suitable linking moiety, and wherein the 5-amino group has been used to form an amide linkage to the remainder of the polyiodinated polyamino compound. In a particular example, the polyiodinated polyamino compound may comprise a residue of a 5-amino-1,3-hydroxyalkyl-substituted-2,4,6-triiodo-1,3-benzenedicarboxamide compound, for instance, a residue of a 5-amino-N,N′-bis(hydroxyalkyl)-2,4,6-triiodo-1,3-benzenedicarboxamide compound, such as a residue of 5-amino-N,N′-bis(2,3-dihydroxypropyl)-2,4,6-triiodo-1,3-benzenedicarboxamide, also known as 5-amino-N,N′-bis(2,3-dihydroxypropyl)-2,4,6-triiodoisophthalamide (CAS #76801-93-9), in which the 5-amino group has been used to form an amide linkage to the remainder of the polyiodinated polyamino compound.

As also previously indicated, in some embodiments, the polyiodinated aromatic moiety of the polyiodinated polyamino compounds may correspond to a residue of an hydroxyl-substituted polyiodinated aromatic compound.

For example, the hydroxyl-substituted polyiodinated aromatic compound may comprise a monocyclic or multicyclic aromatic structure, a plurality of iodine groups, and one or a plurality of hydroxyl groups. For example, in some embodiments, the polyiodinated polyamino compound may comprise a residue of 2,3,5-triiodobenzenemethanol,

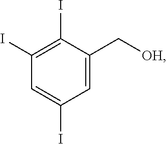 [SMILES: OCc1cc(I)cc(I)c1I]

a residue of 2,3,5-triiodobenzeneethanol,

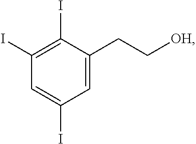 [SMILES: OCCc1cc(I)cc(I)c1I]

a residue of 2,3,5-triiodobenzenepropanol,

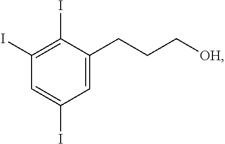 [SMILES: OCCCc1cc(I)cc(I)c1I]

or a residue of iodixanol,

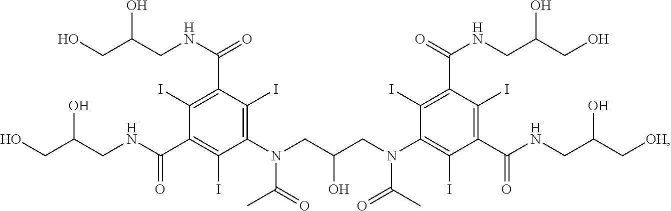 [SMILES: CC(=O)N(CC(O)CN(C(C)=O)c1c(I)c(C(=O)NCC(O)CO)c(I)c(C(=O)NCC(O)CO)c1I)c1c(I)c(C(=O)NCC(O)CO)c(I)c(C(=O)NCC(O)CO)c1I]

among others.

In some aspects, the present disclosure pertains to processes of making polyiodinated polyamino compounds such as those described above.

For example, in an optional first process, an amino-substituted polyiodinated aromatic compound such as one of the amino-substituted polyiodinated aromatic compounds described above may be protected with a suitable protective agent in order to modify the solubility of the amino-substituted polyiodinated aromatic compound for compatibility with other reactants in a subsequent amide coupling reaction (described below). For example, hydroxyl groups of an amino-substituted polyiodinated aromatic compound that comprises a monocyclic or multicyclic aromatic structure that is substituted with an amino group, a plurality of iodine groups, and one or a plurality of hydrophilic functional groups that comprise a C₁-C₄-dihydroxyalkyl group may be protected with 2,2-dimethoxypropane to obtain an acetal-protected amino-substituted polyiodinated aromatic compound.

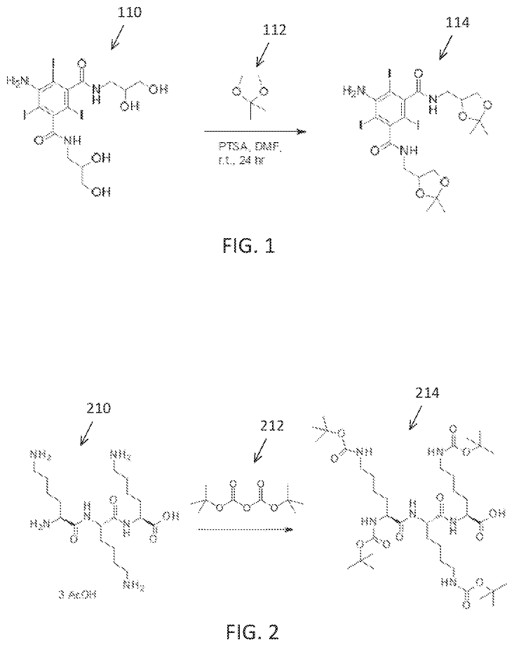

In a particular example shown in FIG. 1, using 5-amino-N,N′-bis(2,3-dihydroxypropyl)-2,4,6-triiodoisophthalamide (110) (CAS #76801-93-9) as the primary iodinated structure, the 2,3-dihydroxypropyl groups of the 5-amino-N,N′-bis(2,3-dihydroxypropyl)-2,4,6-triiodoisophthalamide (110) are protected using 2, 2-dimothoxypropane (112) in the presence of DMF (dimethylformamide) and p-toluenesulfonic acid (PTSA) at room temperature, to obtain an acetal-protected iodinated molecule, 5-amino-N1,N3-bis[(2,2-dimethyl-1,3-dioxolan-4-yl)methyl]-2,4,6-triiodo-benzene-1,3-dicarboxamide (114).

In a second process, a protected carboxyl-substituted polyamino compound in which amino groups of the carboxyl-substituted polyamino compound are protected is formed. Examples of carboxyl-substituted polyamino compounds are described above and include polylysines and various carboxyl-terminated polyamines, with a specific example being trilysine. With reference to FIG. 2, amino groups of trilysine (210) are protected using di-tert-butyl dicarbonate (112), thereby forming tBoc-protected trilysine (214). This leaves the carboxyl group of the protected compound (tBoc-protected trilysine) available for amide coupling.

In a third process, the acetal-protected amino-substituted polyiodinated aromatic compound prepared as described in the first process is coupled with the protected carboxyl-substituted polyamino compound as described in the second process in an amide coupling reaction (e.g., via a carbodiimide coupling reagent) to form a protected polyiodinated polyamino compound. This is followed by deprotection (e.g., under acidic conditions), to form a final polyiodinated polyamino compound.

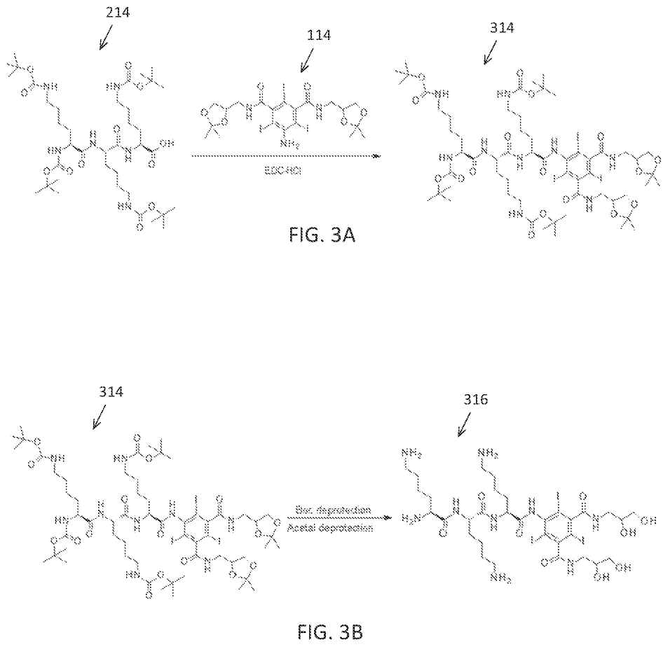

With reference to FIG. 3A, the acetal protected iodinated molecule of FIG. 1, 5-amino-N1,N3-bis[(2,2-dimethyl-1,3-dioxolan-4-yl)methyl]-2,4,6-triiodo-benzene-1,3-dicarboxamide (114), is coupled to the tBoc-protected trilysine (214) of FIG. 2 in the presence of 1-ethyl-3-(3-dimethylaminopropyl)carbodiimide hydrochloride (EDC·HCl) to form a protected polyiodinated polyamino compound (314). Then, as shown in FIG. 3B, the protected polyiodinated polyamino compound (314) is deprotected to form the final polyiodinated polyamino compound (316).

As another example, a hydroxyl-substituted polyiodinated aromatic compound as described above, for example, iodixanol, is coupled with the protected carboxyl-substituted polyamino compound as described above in an ester coupling reaction (e.g., using a carbodiimide coupling reagent) to form a protected polyiodinated polyamino compound. This is followed by deprotection (e.g., under acidic conditions), to form a final polyiodinated polyamino compound.

As noted above, in some aspects of the present disclosure, a radiopaque crosslinked hydrogel is provided that comprises a crosslinked reaction product of (a) a polyiodinated polyamino compound such as those described above and (b) a reactive multi-arm polymer that comprises a plurality of polymer arms that have reactive end groups that are reactive with the amino groups of the polyiodinated polyamino compound. In various embodiments, such crosslinked products are visible on fluoroscopy. In various embodiments, such crosslinked products have a radiopacity that is greater than 250 Hounsfield units (HU), beneficially anywhere ranging from 250 HU to 500 HU to 750 HU to 1000 HU or more. Such crosslinked products may be formed in vivo (e.g., using a delivery device like that described below), or such crosslinked products may be formed ex vivo and subsequently administered to a subject. Such crosslinked products can be used in a wide variety of biomedical applications, including medical devices, implants, and pharmaceutical compositions.

In various embodiments, the reactive end groups of the reactive multi-arm polymer and the amino groups of the polyiodinated polyamino compound react with one another via an amide coupling reaction to form a crosslinked product. The reactive multi-arm polymer may be water soluble.

Reactive multi-arm polymers for use herein include those that comprise a plurality of polymer arms (e.g., having two, three, four, five, six, seven, eight, nine, ten, eleven, twelve or more arms), wherein two or more polymer arms of the multi-arm polymers comprise one or more reactive end groups. In some embodiments, compositions containing the reactive multi-arm polymers may be provided in which a percentage of the polymer arms comprising one or more reactive end groups may correspond to between 50% and 100% of the total number of polymer arms in the composition (e.g., ranging anywhere from 50% to 70% to 80% to 90% to 95% to 99% to 100% of the total number of polymer arms) (in other words, ranging between any two of the preceding numerical values). Typical average molecular weights for the reactive multi-arm polymers for use herein range from 5 to 50 kDa. In various embodiments, the reactive multi-arm polymers for use herein have a melting point of 40° for greater, preferably 45° for greater.

In various embodiments, the polymer arms are hydrophilic polymer arms. Such hydrophilic polymer arms may be composed of any of a variety of synthetic, natural, or hybrid synthetic-natural polymers including, for example, poly(alkylene oxides) such as poly(ethylene oxide) (also referred to as polyethylene glycol or PEG), poly(propylene oxide) or poly(ethylene oxide-co-propylene oxide), poly(vinylpyrrolidone), polyoxazolines including poly(2-alkyl-2-oxazolines) such as poly(2-methyl-2-oxazoline), poly(2-ethyl-2-oxazoline) and poly(2-propyl-2-oxazoline), poly(vinyl alcohol), poly(allyl alcohol), poly(ethyleneimine), poly(allylamine), poly(vinyl amine), poly(amino acids), polysaccharides, and combinations thereof.

In some embodiments, the polymer arms extend from a core region. In certain of these embodiments, the core region comprises a residue of a polyol that is used to form the polymer arms. Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, adonitol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others.

In certain beneficial embodiments, the core region comprises a residue of a polyol that contains two, three, four, five, six, seven, eight, nine, ten or more hydroxyl groups. In certain beneficial embodiments, the core region comprises a residue of a polyol that is an oligomer of a sugar alcohol such as glycerol, mannitol, sorbitol, inositol, xylitol, or erythritol, among others.

In certain embodiments, the reactive end groups may be electrophilic groups selected from imidazole esters, imidazole carboxylates, benzotriazole esters, or imide esters, including N-hydroxysuccinimidyl esters. A particularly beneficial reactive end group is an N-hydroxysuccinimidyl ester group. In certain embodiments, the reactive end groups are linked to the polymer arms via a hydrolysable ester group. For instance, the polymer arms may be terminated with the following reactive, hydrolysable groups, among others: succinimidyl glutarate groups, succinimidyl succinate groups, succinimidyl carbonate groups, or succinimidyl adipate groups.

Further examples of reactive multi-arm polymers are described, for example, in U.S. Patent Application Nos. 2011/0142936, 2021/0061950, 2021/0061954 and 2021/0061957.

In some aspects of the present disclosure, a system is provided that comprises (a) a first composition that comprises a polyiodinated polyamino compound as described hereinabove and (b) a second composition that comprises a reactive multi-arm polymer as described hereinabove. Such systems can be used to form crosslinked hydrogels, either in vivo or ex vivo.

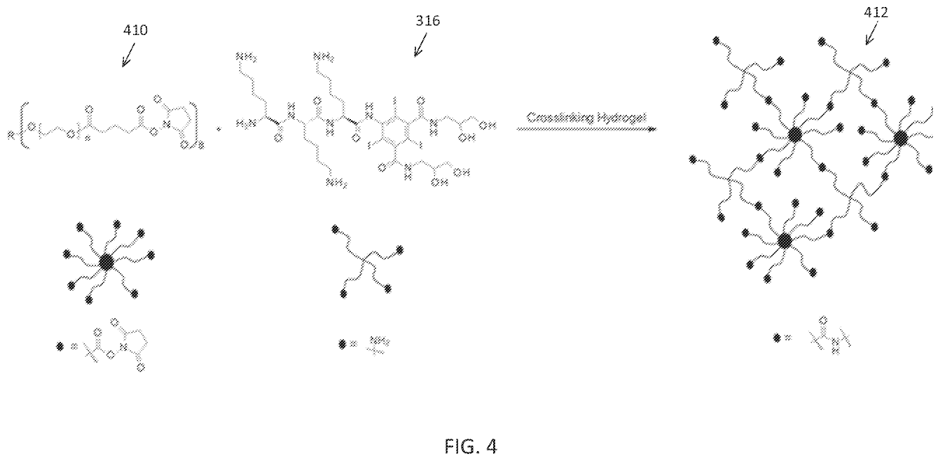

For example, as shown schematically in FIG. 4, a second composition comprising a reactive multi-arm polymer (410) like that described above, which comprises a core region and a plurality of hydrophilic polymer arms having reactive end groups (i.e., succinimidyl glutarate groups) (where R is a core region, such as a polyol residue, and n ranges, for example, from 15 to 45) can be crosslinked with a first composition comprising a polyiodinated polyamino compound (316) like that described above, which comprises amino groups that are reactive with the reactive groups (i.e., succinimidyl glutarate groups) of the reactive multi-arm polymer (410), to form a crosslinked product 412, which may be in the form of a hydrogel when hydrated.

The first composition may be a first fluid composition comprising the polyiodinated polyamino compound or a first dry composition that comprises the polyiodinated polyamino compound, to which a suitable fluid such as water for injection, saline, etc. can be added to form a first fluid composition. In addition to the polyiodinated polyamino compound, the first composition may further comprise additional agents such as those described below.

The second composition may be a second fluid composition comprising the reactive multi-arm polymer or a second dry composition that comprises the reactive multi-arm polymer, to which a suitable fluid such as water for injection, saline, etc. can be added to form a second fluid composition). In addition to the reactive multi-arm polymer, the second composition may further comprise additional agents such as those described below.

In various embodiments, the system will include one or more delivery devices for delivering the first and second compositions to a subject. For example, the system may include a delivery device that comprises a first reservoir that contains the first composition (e.g., a first fluid composition or a first dry composition to which a suitable fluid can be added to form the first fluid composition) and a second reservoir that contains the second composition (e.g., a second fluid composition or a second dry composition to which a suitable fluid such as water for injection, saline, etc. can be added to form the second fluid composition). During operation, the first and second compositions are dispensed from the first and second reservoirs, whereupon the first and second compositions interact and crosslink with one another to form a hydrogel.

In particular embodiments, the system may include a delivery device that comprises a double-barrel syringe, which includes first barrel having a first barrel outlet, which first barrel contains the first composition, a first plunger that is movable in first barrel, a second barrel having a second barrel outlet, which second barrel contains the second composition, and a second plunger that is movable in second barrel.

In some embodiments, the device may further comprise a mixing section having a first mixing section inlet in fluid communication with the first barrel outlet, a second mixing section inlet in fluid communication with the second barrel outlet, and a mixing section outlet. In some embodiments, the device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions interact and crosslink to form a hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

In some embodiments, the first composition comprising the polyiodinated polyamino compound, the second composition comprising the reactive multi-arm polymer, or the crosslinked product of the polyiodinated polyamino compound and the reactive multi-arm polymer, may include one or more additional agents. Examples of such additional agents include therapeutic agents, and further imaging agents (beyond the iodine groups that are present in the polyiodinated polyamino compound).

Examples of further imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd^{(III)}, Mn^{(II)}, Fe^{(III)} and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) radiocontrast agents, such as those based on the clinically important isotope ^{99m}Tc, as well as other gamma emitters such as ¹²³I, ¹²⁵I, ¹³¹I, ¹¹¹In, ⁵⁷Co, ¹⁵³Sm, ¹³³Xe, ⁵¹Cr, ^{81m}Kr, ²⁰¹TI, ⁶⁷Ga, and ⁷⁵Se, among others, (e) positron emitters, such as ¹⁸F, ¹¹C, ¹³N, ¹⁵O, and ⁶⁸Ga, among others, may be employed to yield functionalized radiotracer coatings, and (f) contrast agents for use in connection with near-infrared (NIR) imaging, which can be selected to impart near-infrared fluorescence to the coatings of the present disclosure, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others. NIR-sensitive dyes include cyanine dyes, squaraines, phthalocyanines, porphyrin derivatives and borondipyrromethane (BODIPY) analogs, among others.

Crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

What is claimed is:

1. A system for forming a hydrogel that comprises (a) a first composition that comprises a polyiodinated polyamino compound that comprises a polyamino moiety linked to a polyiodinated aromatic moiety by an amide group and (b) a second composition that comprises a reactive multi-arm polymer that comprises a plurality of hydrophilic polymer arms having reactive end groups that are reactive with amino groups of the polyiodinated polyamino compound.

2. The system of claim 1, wherein the polyiodinated aromatic moiety comprises a residue of an amino-substituted polyiodinated aromatic compound.

3. The system of claim 1, wherein the polyiodinated aromatic moiety comprises a residue of an amino-substituted polyiodinated aromatic compound that comprises a monocyclic or multicyclic aromatic moiety that is substituted with (a) an amino group, (b) a plurality of iodine groups and (c) one or a plurality of hydrophilic functional groups.

4. The system of claim 1, wherein the polyiodinated aromatic moiety comprises a monocyclic or multicyclic aromatic moiety that is substituted with (a) a plurality of iodine groups and (b) one or a plurality of hydrophilic functional groups.

5. The system of claim 4, wherein the monocyclic or multicyclic aromatic moiety is selected from benzene and naphthalene and wherein the hydrophilic functional groups comprise hydroxyalkyl groups.

6. The system of claim 1, wherein the polyiodinated aromatic moiety is a 1,3-substituted-2,4,6-triiodobenzene moiety in which a substituent at each of the 1- and 3-positions comprises a dihydroxyalkyl group.

7. The system of claim 1, wherein the polyamino moiety comprises a residue of a carboxyl-substituted polyamino compound that comprises a carboxyl group and the polyamino moiety.

8. The system of claim 6, wherein the carboxyl-substituted polyamino compound is selected from a polylysine compound, a carboxyl-substituted carboxyl-terminated poly(allyl amine) compound, a carboxyl-terminated polyvinylamine compound, and a carboxyl-terminated chitosan compound.

9. The system of claim 1, wherein the polyamino moiety comprises a plurality of —(CH₂)ₓ—NH₂ groups where x is 0, 1, 2 3, 4, 5 or 6.

10. The system of claim 9, wherein the plurality of —(CH₂)ₓ—NH₂ groups are disposed along a polymeric moiety.

11. The system of claim 1, wherein the hydrophilic polymer arms comprise one or more hydrophilic monomers selected from ethylene oxide, N-vinyl pyrrolidone, oxazolines, hydroxyethyl acrylate, hydroxyethyl methacrylate, PEG methyl ether acrylate, PEG methyl ether methacrylate, or PNIPAAM.

12. The system of claim 1, wherein the reactive end groups are linked to the hydrophilic polymer arms by a hydrolysable ester and/or wherein the reactive end groups are electrophilic groups.

13. The system of claim 12, wherein the electrophilic groups are selected from imidazole esters, imidazole carboxylates, benzotriazole esters, or imide esters.

14. The system of claim 1, further comprising a delivery device.

15. The system of claim 14, wherein the delivery device comprises a first reservoir that contains the first composition and a second reservoir that contains the second composition, and wherein during operation the first and second compositions are dispensed from the first and second reservoirs, whereupon the first and second compositions interact and crosslink with one another to form the hydrogel.

16. The system of claim 15, wherein the first and second reservoirs comprise syringe barrels.

17. A medical hydrogel formed by reaction of the first composition and the second composition of the system claim 1.

18. A method of making a medical hydrogel comprising reacting (a) a first composition that comprises a polyiodinated polyamino compound that comprises a polyamino moiety linked to a polyiodinated aromatic moiety by an amide group with (b) a second composition that comprises a reactive multi-arm polymer that comprises a plurality of hydrophilic polymer arms having reactive end groups that are reactive with amino groups of the polyiodinated polyamino compound.

19. A method of making a polyiodinated polyamino compound comprising (a) forming an amide linkage between an amino group of an amino-substituted polyiodinated aromatic compound and a carboxyl group of a protected carboxyl-substituted polyamino compound in which the amino groups are protected and (b) the deprotecting the protected amino groups.

20. The method of claim 19, wherein the amino-substituted polyiodinated aromatic compound is a protected amino-substituted polyiodinated aromatic compound that comprises a monocyclic or multicyclic aromatic moiety that is substituted with (a) the amino group, (b) a plurality of iodine groups and (c) one or a plurality of hydrophilic functional groups that comprise an acetal-protected dihydroxyalkyl group.
